(19) United States
(12) Patent Application Publication
(10) Pub. No.: US 2007/0253995 A1
(43) Pub. Date: Nov. 1, 2007

(54) Drug Delivery Methods and Devices for Treating Stress Urinary Incontinence

(75) Inventor: Keith R. Hildebrand, Houlton, WI (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 11/380,919

(22) Filed: Apr. 28, 2006

(51) Int. Cl. A61K 31/445; A61K 31/381; A61K 31/4184; A61K 31/4178; A61K 31/137; A61K 31/343

(52) U.S. Cl. 424/422; 514/469; 514/438; 514/397; 514/394; 514/649; 514/317

(57) ABSTRACT

The disclosure describes a method and system for delivering a drug to the Onuf's nucleus of a patient for the treatment or prevention of stress urinary incontinence. The system includes drug delivery devices that deliver one or more drugs to a site located adjacent to, around or within the Onuf's nucleus stress incontinence alleviation. A depot configured to release a therapeutically effective amount of a stress incontinence-reducing drug is one drug delivery device disclosed. Other drug delivery devices include implantable infusion pumps.

### FIELD OF THE INVENTION

This invention relates to drug delivery methods and devices for treating or preventing stress urinary incontinence.

### BACKGROUND OF THE INVENTION

Stress incontinence, the most common type of incontinence, is the unacceptable passage of urine under the stress of increased abdominal pressure. This increased pressure typically results from sneezing, laughing, coughing, lifting, or other activity that puts pressure on the bladder. Stress incontinence involves pathology associated with the urethra and periurethral structures as opposed to the bladder. It is most common in post-menopausal women and women who have had multiple children. Sudden increases in intra-abdominal pressure are passively transmitted to the bladder and urine leakage occurs because of inadequate urethral closure mechanisms.

A range of therapies are currently used to manage and treat stress incontinence. One conservative treatment method is the use of pelvic-floor exercises, also called Kegel exercises that are designed to strengthen the striated skeletal muscles of the pelvic which support and maintain proper orientation of the lower urinary tract as well as provide additional closure forces to the urethra. Such exercise therapy may be ineffective for a number of patient populations, such as the elderly, obese, physically impaired, or patients who lack the motivation to continue a long-term exercise program.

Certain orally administered drugs been also been used to treat stress incontinence. For example alpha-adrenergic agonists such as commonly used nasal decongestants (e.g., pseudoephedrine and phenylpropanolamine) have been used to treat stress incontinence but their use is limited in many patients because of potentially serious side effects which include increases in blood pressure and central nervous system stimulation. Another class of compounds that have been used with some success in the treatment or prevention of stress incontinence includes compounds which affect the reuptake of monoamine neurotransmitters such as norepinephrine and serotonin. Recently, Phase III clinical trial results have provided evidence that an oral formulation of duloxetine is effective for treating women with stress urinary incontinence. The use of the oral formulation did produce significant side effects in some patients. Most of the side effects were centrally mediated (i.e. at the brain level) and included: nausea, headache, insomnia, dry mouth, dizziness and fatigue which generally disappeared within 1 to 4 weeks of therapy initiation.

### SUMMARY OF THE INVENTION

In general, the invention is directed to techniques for treating or preventing stress urinary incontinence in a patient comprising delivering a drug delivery device adapted to administer at a controlled rate a stress incontinence-reducing amount of a drug that prevents or treats urinary incontinence to motor neurons located within an Onuf's nucleus of a patient. In one embodiment, the drug delivery device is a depot. Desirably, the depots are configured to release therapeutic agents over a period of time, e.g. up to a month or two, and to closely mimic implantable infusion device delivery profile, i.e. small amounts of therapeutic agent over a sustained period.

Stress incontinence-reducing drugs useful in the method and device of the invention includes drugs that may be administered to a subject at a dose effective to treat or prevent stress urinary incontinence as determined by well known means without significant side effects. Such drugs include selective inhibitors of the reuptake of both serotonin and norepinephrine, for example, venlafaxine and duloxetine, selective inhibitors of the reuptake of serotonin, for example, paroxetine, clomipramine, fluoxetine, sertraline, and citalopram, selective inhibitors of the reuptake of norepinephrine, such as desipramine, nortriptyline, imipramine, and protryptyline, alpha 1-adrenergic agonists, such as oxymetazoline, phenylephrine or pseudoephedrine and 5-HT2 receptor agonists (provided the agonist is not 1-[6-chloro-5-(trifluoromethyl)-2-pyridinyl]piperazine (Org-12962), such as m-CPP, MK212, Ro-60-0175, WAY-161503, YM-348 and the pharmaceutically acceptable salts thereof.

Various embodiments described in this disclosure may provide one or more advantages over existing methods, devices and systems used to alleviate chronic pelvic pain.

### BRIEF DESCRIPTION OF DRAWINGS

FIG. 1 is a diagrammatic illustration of a spinal column of a human.

FIG. 2 is diagrammatic illustration of a cross section of the sacral spinal cord showing the location of Onuf's nucleus and the relationship of the sacral spinal cord to the bladder.

FIG. 3 is a diagrammatic illustration of an infusion system that may be useful in the method of the invention.

FIG. 4 is a diagrammatic illustration of an infusion device and a catheter implanted in a patient.

The figures are not necessarily to scale.

### DETAILED DESCRIPTION OF THE INVENTION

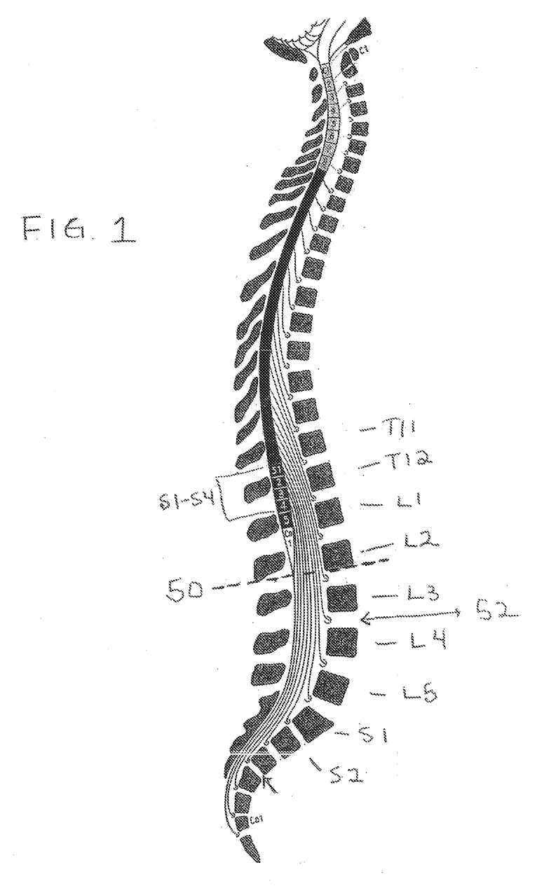

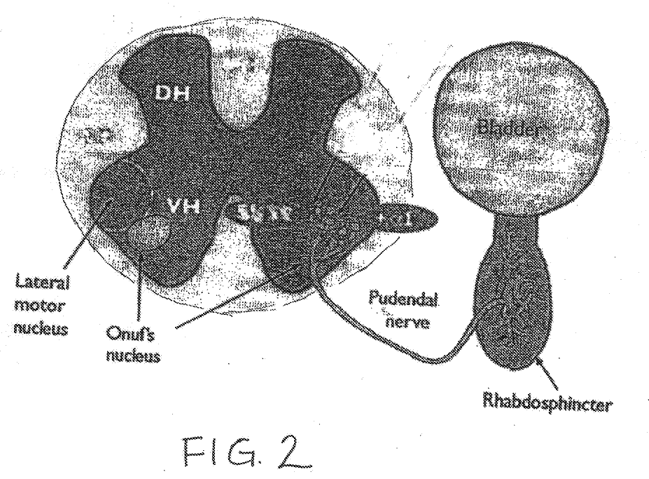

The present invention relates to methods and devices for decreasing, eliminating, or managing stress incontinence by administering a therapeutically effective amount of stress incontinence-reducing drug to a site adjacent to, around or within the Onuf's nucleus of the patient. FIG. 1 is a schematic diagram illustrating the numbered segments vertebrae forming the spinal column. Onuf's nucleus is located in the ventral horn of the spinal cord between segments S1-S4, which corresponds to a vertebral level of about from T11 to L1. The dotted line shown in FIG. 1 as 50 illustrates the approximate location of where the spinal cord ends. FIG. 2 illustrates contains the cell bodies of the somatic motor neurons which innervate the striated rhabdosphincter shown on FIG. 2 by way of the pudendal nerve also shown on FIG. 2. It has been found that Onuf's nucleus plays a key role in urinary continence and the micturition reflex. It is believed that the motor neurons in this nucleus receive both cortical inputs as well as noradrenergic and serotonergic input from various brain stem structures, including the PONS.

As used herein, the term “stress incontinence” shall refer to stress urinary incontinence.

For the purposes of this disclosure, the term “drug” means any pharmacological or therapeutic agent or combination of agents delivered to provide therapy to a patient (human or non-human animal). The drugs will typically be liquids or materials contained in liquid carriers as either solutions or mixtures (although where used herein, the term “solution” refers to both solutions and mixtures).

As used herein the term “therapeutically effective amount” means an amount of a drug which is effective to achieve a desired therapeutic effect, e.g., alleviation of chronic pelvic pain. The precise desired therapeutic effect (e.g., the degree of pain relief, the cause of the pain relief, etc.) will vary according to the condition to be treated and a variety of other factors that are known by those of ordinary skill in the art.

It will be understood that the type and amount of stress incontinence-reducing drug delivered may be altered based upon the response of a patient to the drug. Any indication that the patient's stress urinary incontinence is improving or worsening is an indication that modification may be appropriate. Such determinations can be readily made by, for example, a physician attending to the subject's care and in the case of stress urinary incontinence is often determined in part by the decrease in frequency of incontinence episodes. Quality of life instruments such as the Incontinence Quality of Life questionnaire and the SF-36 can also be used in making the assessment

In an embodiment of the invention, the effective amount to treat or prevent stress urinary incontinence will be the amount of drug required to reduce the frequency of incontinence episodes by an amount of at least about 25% and desirably by an amount of at least about 50% without the subject experiencing side effects that significantly diminish the subject's quality of life.

The amount of the stress incontinence-reducing drug may also be adjusted based upon the presentation and severity of side effects in a patient. Side effects may be recognizable by the patient, a physician attending to the care of the patient, other health care professionals, and the like. A physician or other health care professional may adjust therapy parameters based on side effects. Side effects which may be associated with some stress incontinence-reducing drugs useful with the method of the invention include nausea, dizziness, dry mouth, constipation, insomnia, somnolence, and asthenia. For certain drugs, such as the alpha 1-adrenergic agonists, such as oxymetazoline, phenylephrine or pseudoephedrine, the side effects may also include rapid heartbeat, anxiety or agitation and increased blood pressure.

Effective dosages for use in methods as described herein can be determined by those of skill in the art, particularly when effective systemic dosages are known for a particular therapeutic agent. Dosages may typically be decreased by at least 90% of the usual systemic dose if the therapeutic agent is provided in a targeted fashion. In other embodiments, the dosage is at least 75%, at least 80% or at least 85% of the usual system dose for a given condition and patient population. Dosage is usually calculated to deliver a minimum amount of one or more therapeutic agent per day, although daily administration is not required. If more than one pharmaceutical composition is administered, the interaction between the same is considered and the dosages calculated.

In one embodiment of this invention, prior to implanting a drug delivery device of the invention that will administer drug for a sustained period of time, the subject's physician may wish to evaluate the suitability of a particular stress incontinence-reducing drug. To do so, the physician may deliver a bolus of therapeutically effective amount of the drug candidate and determine whether the patient experiences any side effects from the drug. If the injection of drug at the site ameliorates the patient's symptoms, a physician may conclude that sustained administration over a period of days, weeks or months may be efficacious, and may proceed to surgically implant or otherwise administer drug delivery devices in accordance with the invention.

As used herein a “controlled administration system” is a direct administration system to deliver stress incontinence-reducing drugs to a site within the subject's spinal column adjacent to, around, or within the Onuf's nucleus, and includes, but is not limited to, a depot, an osmotic pump, infusion pump, implantable mini-pumps, a peristaltic pump, other drug pumps, or a system administered locally by insertion of a catheter at or near the target site, the catheter being operably connected to a drug delivery pump. It is understood that pumps can be internal or external as appropriate.

In one embodiment, the drug delivery device is a depot. Suitable depots may take the form of capsules, microspheres, particles, rods, gels, coatings, matrices, wafers, pills, and the like. A depot may comprise a biopolymer. The biopolymer may be a sustained-release biopolymer and may be biodegradable. The depot may be deposited at or near, generally in close proximity, to a target site, such as a spinal or perispinal location. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof. Depots that may be useful to deliver chronic pelvic pain-alleviating drugs of the invention include without limitation those described in Chen et al. U.S. Published Application No. 2005/0079202, U.S. Pat. No. 5,980,927 and in co-pending U.S. application being filed on even date herewith entitled: “SCREENING CANDIDATES FOR IMPLANTABLE INFUSION DEVICES” the teachings of each of the above is incorporated herein by reference in its entirety.

It is desirable that the drug delivery device be able to accurately, precisely and reliably deliver the intended amount of drug over the intended period of time. As there are relatively inexpensive alternatives to the use of drugs for the treatment of stress incontinence, any drug may seem expensive to a subject and those drugs formulated to retain stability and efficacy over extended periods of time are likely to be some of the most expensive, although the ability to obtain relief from frequent incontinence episodes for long periods of time may be attractive to subjects who experience stress incontinence. Thus, the ability to efficiently formulate, process, package and deliver the drugs delivered via the controlled administration system with minimal loss of drug stability and efficacy is desirable. In a preferred embodiment, compositions suitable for controlled administration systems of the instant invention will be carefully formulated for the desired therapeutic effect. The drug itself may be on a continuum of rapid-acting to long-acting or it may be formulated to a continuum of rapid release or sustained release. Still further, the options for delivery of a therapeutically effective amount of a chronic pelvic pain-alleviating drug on a continuum and includes but is not limited to rapid and repeating delivery at intervals ranging to continuous delivery. Delivery may occur at a desired site over a desired period of time for adequate distribution and absorption in the patient.

Controlled administration systems that may be useful with the method of the invention includes, for example, an infusion pump that administers a stress incontinence-reducing drug through a catheter the proximal end of which is near the Onuf's nucleus, an implantable mini-pump, an implantable controlled release device (such as, for example, the device described in U.S. Pat. No. 6,001,386), and a sustained release delivery system (such as the system described in U.S. Pat. No. 6,007,843).

In one embodiment of the invention, the stress incontinence-reducing drug or a depot comprising such a drug is delivered to a site located adjacent to, around, or within the Onuf's nucleus, using a drug delivery device comprising a drug delivery catheter having a proximal end and a distal end, the proximal end having an opening to deliver a drug in situ.

In yet another embodiment of the invention, a drug delivery catheter that is a component of an implantable infusion system may be used to deliver the stress incontinence-reducing drug or depot comprised of such drug to the area around the Onuf's nucleus. Any implantable infusion system may be used in accordance with the teachings of this disclosure. Implantable infusion systems typically comprise an infusion device and a catheter operably coupled to the infusion device. The therapy delivery device may be a pump device. Non-limiting examples of pump devices include osmotic pumps, fixed-rate pumps, programmable pumps and the like. Each of the aforementioned pump systems comprise a reservoir for housing a fluid composition comprising a therapeutic agent. The catheter comprises one or more delivery regions, through which the fluid may be delivered to one or more target regions of the subject.

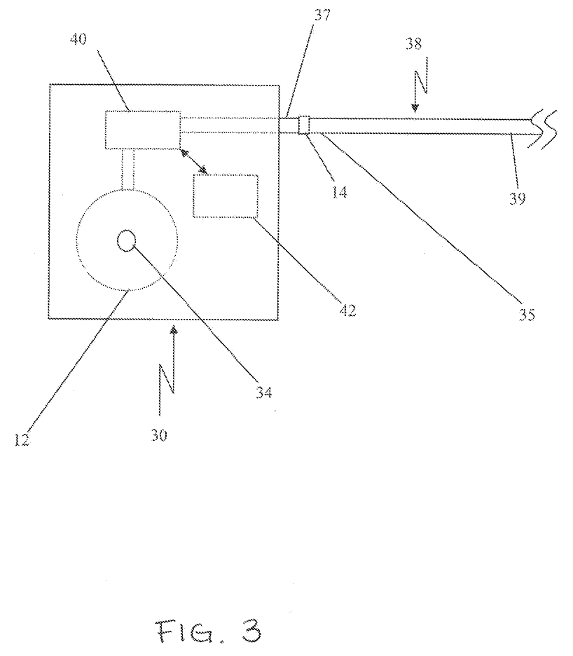

The infusion device 30 shown in FIG. 3 comprises a reservoir 12 for housing a composition comprising a therapeutic agent and a pump 40 operably coupled to the reservoir 12. The catheter 38 shown in FIG. 3 has a proximal end 35 coupled to the therapy delivery device 30 and a distal end 39 adapted to be implanted in a patient. Between the proximal end 35 and distal end 39 or at the distal end 39, the catheter 38 comprises one or more delivery regions (not shown) through which the therapeutic agent may be delivered. The infusion device 30 may have a port 34 into which a hypodermic needle can be inserted to inject a quantity of therapeutic agent into reservoir 12. The infusion device 30 may have a catheter port 37, to which the proximal end 35 of catheter 38 may be coupled. The catheter port 37 may be operably coupled to reservoir 12. A connector 14 may be used to couple the catheter 38 to the catheter port 37 of the therapy delivery device 30. The infusion device 30 may be operated to discharge a predetermined dosage of the pumped fluid into a target region of a patient. The infusion device 30 may contain a microprocessor 42 or similar device that can be programmed to control the amount of fluid delivery. The programming may be accomplished with an external programmer/control unit via telemetry. A controlled amount of fluid comprising a therapeutic agent may be delivered over a specified time period. With the use of a programmable delivery device 30, different dosage regimens may be programmed for a particular patient. Additionally, different therapeutic dosages can be programmed for different combinations of fluid comprising therapeutics. Those skilled in the art will recognize that a programmed infusion device 30 allows for starting conservatively with lower doses and adjusting to a more aggressive dosing scheme, if warranted, based on safety and efficacy factors.

If it is desirable to administer more than one therapeutic agent, the fluid composition within the reservoir 12 may contain a second, third, fourth, etc. therapeutic agent. Alternatively, the device 30 may have more than one reservoir 12 for housing additional compositions comprising a therapeutic agent. When the device 30 has more than one reservoir 12, the pump 40 may draw fluid from one or more reservoirs 12 and deliver the drawn fluid to the catheter 38. The device 30 may contain a valve operably coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the pump 40. A catheter 38 may have more than one lumen. Each lumen may be adapted to deliver a therapeutic agent from one or more reservoirs 12 of the device 30. It will also be understood that more than one device 30 may be used if it is desirable to deliver more than one therapeutic agent. Such therapy delivery devices, catheters, and systems include those described in, for example, copending application Ser. No. 10/746,269, entitled IMPLANTABLE DRUG DELIVERY SYSTEMS AND METHODS, filed on Dec. 23, 2003, which application is hereby incorporated herein by reference.

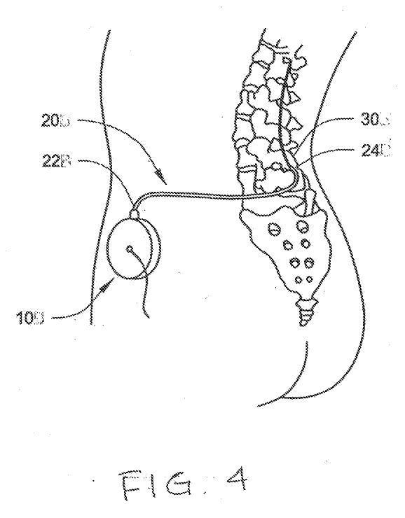

FIG. 4 is a schematic diagram of a drug delivery system that includes an infusion pump assembly 10 and catheter 20 having a proximal end 22 implanted within the patient.

As used in a method of this invention, the proximal end 22 of a drug delivery catheter (including without limitations drug delivery catheters that are not connected to drug pumps) may be inserted into the spinal region level below the end of the spinal cord such as location indicated by arrow 52 in FIG. 1, then threaded up to the desired level which as shown in FIG. 1 would be in the area between S1-S4 where Onuf's nucleus is located. Many catheters are very limp, making it difficult or impossible to steer the catheter into a desired specific area of the spinal region. A guidewire may be used to stiffen the catheter during placement. The guidewire is then typically removed before the catheter is used to administer drugs.

The preceding specific embodiments are illustrative of the practice of the invention. It is to be understood, therefore, that other expedients known to those skilled in the art or disclosed herein may be employed without departing from the invention or the scope of the appended claims.

What is claimed is:

1. A method for treating or preventing stress urinary incontinence in a mammalian subject comprising administering a therapeutically effective amount of stress incontinence-reducing drug in the subject's spinal canal at a site adjacent to, around or within an Onuf's nucleus.

2. The method of claim 1, wherein a depot is used to administer the drug.

3. The method of claim 1, wherein the drug is a selective inhibitor of the reuptake of both serotonin and norepinephrine.

4. The method of claim 3, wherein the drug is venlafaxine.

5. The method of claim 3, wherein the drug is duloxetine.

6. The method of claim 1 wherein the drug is a selective inhibitor of the reuptake of serotonin.

7. The method of claim 6, wherein the drug is selected from the group consisting of paroxetine, clomipramine, fluoxetine, sertraline, and citalopram.

8. The method of claim 1 wherein the drug is a selective inhibitor of the reuptake of norepinephrine.

9. The method of claim 8, wherein the drug is selected from the group consisting of desipramine, nortriptyline, imipramine, and protryptyline.

10. The method of claim 1, wherein the drug is a 5-HT2 receptor agonist selected from the group consisting of m-CPP, MK212, Ro-60-0175, WAY-161503 and YM-348.

11. The method of claim 11, wherein the drug is an alpha 1-adrenergic agonist.

12. The method of claim 11, wherein the alpha 1-adrenergic agonist is oxymetazoline, phenylephrine or pseudoephedrine.

13. The method of claim 1, wherein administration is localized and sustained.

14. The method of claim 1, wherein the administration occurs over a period of from about at least one day to about three months.

15. The method of claim 1, wherein the administration is continuous.

16. The method of claim 1, wherein the administration is periodic.

17. The method of claim 1, wherein the drug has a controlled release rate.

18. The method of claim 17, wherein the controlled release rate is from about 24 hours to about 31 days.

19. The method of claim 17, wherein the controlled release rate is from about at least one day to about three months.

20. The method of claim 1, wherein the stress incontinence drug is administered via a drug delivery device.

21. The method of claim 20, wherein the drug delivery device is implanted in the patient adjacent to, around, or within the Onuf's nucleus.

22. The method of claim 1, wherein the stress incontinence drug is administered via a drug delivery device, where the drug delivery comprises a catheter having a proximal end and a distal end, the proximal end having an opening to deliver a drug in situ, the distal end being fluidly connected to a drug delivery pump, and the method further comprising inserting the catheter adjacent to, around, or within the Onuf's nucleus.

23. The method of claim 22, wherein the pump is implanted in the subject.

24. The method of claim 22, wherein the pump is externally attached to the subject.

25. The method of claim 22, wherein a depot comprising the stress incontinence-reducing drug is contained in the pump.

26. The method of claim 20, wherein the drug delivery device comprises a drug delivery catheter having a proximal end and a distal end, the proximal end having an opening to deliver a drug in situ and the therapeutically effective amount of the stress incontinence-reducing drug is administered via the catheter.

27. The method of claim 27, wherein the stress incontinence-reducing drug is in a depot administered via the catheter.

28. A device for administering a stress incontinence-reducing drug to a subject at a sustained rate over a period of time, the device being shaped, sized and adapted for delivering the device into the spinal region of the subject adjacent to, around, or within the Onuf's nucleus, the device comprising a biostable polymeric body, wherein the chronic pelvic pain alleviating-drug is loaded into the body or dissolved or dispersed in the polymer in an amount sufficient so that a therapeutically effective amount of the drug will be delivered for a predetermined duration of time after implant.

29. The device of claim 28, wherein the predetermined duration of time is from about 24 hours to about 31 days.

30. The device of claim 28, wherein the predetermined duration of time is about 1 week to about three months.

31. The device of claim 28, wherein the predetermined duration of time is about 2 weeks to about six months.

32. The device of claim 28 wherein the stress incontinence-reducing drug is a selective inhibitor of the reuptake of both serotonin and norepinephrine.

33. The device of claim 32 wherein the drug is duloxetine.

34. A system for administering a stress incontinence-reducing drug to a patient in need thereof, the system comprising a controlled administration system for providing controlled and directed delivery of a therapeutically effective amount of the stress incontinence-reducing drug adjacent to, around, or within an Onuf's nucleus within the patient's spinal region.

35. The system of claim 34, wherein the controlled administration system comprises a catheter having a proximal end and a distal end, the proximal end having an opening to deliver a drug in situ, the distal end being fluidly connected to a drug pump.

36. The system of claim 34 wherein the stress incontinence-reducing drug is contained in a depot within the pump.
